(19) United States
(12) Patent Application Publication
(10) Pub. No.: US 2021/0321594 A1
(43) Pub. Date: Oct. 21, 2021

(54) DLGAP2 AS A THERAPEUTIC TARGET FOR AND ALZHEIMER'S DISEASE AND AGE-RELATED COGNITIVE DECLINE

(71) Applicant: The Jackson Laboratory, Bar Harbor, ME (US)

(72) Inventors: Catherine Kaczorowski, Bar Harbor, ME (US); Sarah M. Neuner, Bar Harbor, ME (US)

(73) Assignee: The Jackson Laboratory, Bar Harbor, ME (US)

(21) Appl. No.: 17/245,431

(22) Filed: Apr. 30, 2021

Related U.S. Application Data
(63) Continuation of application No. PCT/US2019/059311, filed on Nov. 1, 2019.
(60) Provisional application No. 62/754,486, filed on Nov. 1, 2018.

(51) Int. Cl. A01K 67/027; A61K 45/06

(52) U.S. Cl. CPC A01K 67/0275; A01K 2227/105; A01K 2267/0312; A61K 45/06

(57) ABSTRACT

Provided herein, in some embodiments, are methods for modulating expression and/or activity of disks large-associated protein 2 (DLGAP2), as well as methods of treating age-related cognitive decline, such as Alzheimer's disease.

### RELATED APPLICATION

This application claims the benefit under 35 U.S.C. § 119(e) of U.S. provisional application No. 62/754,486, filed Nov. 1, 2018, which is incorporated by reference herein in its entirety.

### FEDERALLY SPONSORED RESEARCH

This invention was made with government support under Grant Nos. R01AG054180, F31AG050357, K01AG049164, and R01AG059716 awarded by National institutes of Health. The government has certain rights in the invention.

### BACKGROUND

Aging is the leading risk factor for a number of disorders, including Alzheimer's disease (AD) and other dementia. The mechanisms that underlie aging and age-related cognitive decline remain poorly understood; however, research suggests genetics play a role in susceptibility [1-4]. Identifying the precise genetic factors involved in age-related cognitive decline would provide insight into mechanisms underlying increased susceptibility to AD and other dementia.

### SUMMARY

Genetically diverse mouse models provide a resource to identify genes involved in mediating susceptibility to age-related cognitive decline. Results may inform human studies and enable prioritization of otherwise uninvestigated gene variants. While there are a number of factors that have complicated the identification of genes involved in age-related cognitive decline in human populations, including complex genomes, uncontrolled environmental variables, and limited sample sizes, the mouse represents a critical resource through which to overcome a number of these variables, namely through almost unlimited sample size, well-controlled environmental conditions, and well-defined genetic backgrounds.

Recent efforts to expand and improve mouse genetic resources have produced the Collaborative Cross (CC) and corresponding Diversity Outbred (DO) panels [5-9]. These series of recombinant inbred and outbred mice, respectively, are derived from an 8-parent population segregating for approximately 40 million variants [10]. As this number of genetic variants parallels that observed in the human population, the resulting CC and DO progeny provide unprecedented precision and diversity for systems genetic analysis of complex traits such as age-related cognitive decline.

The present disclosure provides data from quantitative trait loci (QTL) mapping used to identify genomic regions modifying working memory decline in DO mice. The data was also compared to data from human studies in order to evaluate the translational relevance of the findings. From these analyses, disks large-associated protein 2 (DLGAP2) was identified as a cross-species mediator of age-related cognitive decline and Alzheimer's disease (AD).

Thus, some aspects of the present disclosure provide methods comprising delivering to a subject an agent that modulates (e.g., increases or decreases) DLGAP2 expression and/or activity, wherein the subject has symptoms of age-related cognitive decline.

In some embodiments, the subject is a human subject. In some embodiments, the human subject has Alzheimer's disease (AD). In some embodiments, the subject has a modification in a DLGAP2 gene. In some embodiments, the modification is a single nucleotide polymorphism, for example, rs34130287C.

Some aspects of the present disclosure provide methods comprising assaying a subject with symptoms of age-related cognitive decline for the presence or absence of a modification in a DLGAP2 gene (e.g., a SNP, such as rs34130287C), and optionally delivering to the subject an agent that modulates (e.g., increases or decreases) DLGAP2 expression and/or activity.

Other aspects of the present disclosure provide methods comprising administering to a Dlgap2 mutant mouse a candidate agent that modulates DLGAP2 expression and/or activity, and optionally assaying the mouse for an improvement in a symptom of age-related cognitive decline and/or assaying the mouse for an adverse effect.

In some embodiments, the agent is delivered in an amount effective to alleviate the symptoms of the age-related cognitive decline. In some embodiments, the agent is delivered in an amount effective to slow or stop progression of the age-related cognitive decline.

In some embodiments, the agent is selected from polypeptides, polynucleotides, small molecule drugs.

Still other aspects of the present disclosure provide methods comprising delivering to a subject an agent that modulates expression of, or increases activity of, a product encoded by a pathway gene upstream from or downstream from DLGAP2, wherein the subject has symptoms of age-related cognitive decline.

Further aspects of the present disclosure provide methods comprising contacting a neuronal cell that expresses DLGAP2 with an agent that modulates DLGAP2 expression and/or activity.

In some embodiments, an agent increases DLGAP2 expression and/or activity.

### BRIEF DESCRIPTION OF THE DRAWINGS

FIGS. 1A-1E show that Dlgap2 mediates cognitive function across the lifespan in Diversity Outbred (DO) mice. (FIG. 1A) DO mice are a genetically diverse population derived from 8 parental lines, segregating for a total of 40 million single nucleotide polymorphisms. (FIG. 1B) Working memory was assessed on the T-maze at either 6, 12, or 18 months across 487 DO mice (6m=66F/67M, 12m=102F/96M, 18m=76F/80M). All groups performed above chance (50%), and the data was log transformed for subsequent genetic mapping (FIG. 1C). A significant effect of age was observed [F(2, 284)=3.2, p=0.04]. (FIG. 1D) Genetic mapping identified a quantitative trait locus (QTL) on chromosome 8 that significantly interacted with age to mediate working memory performance across the lifespan (LOD=12.5, 1.5 LOD interval=14.3-14.6 Mb). (FIG. 1E) A single protein-coding gene, Dlgap2, was found to be located within the QTL interval, along with a number of regulatory elements.

FIGS. 2A-2D show that increased density of hippocampal long spines positively correlate with cognitive resilience in aging DO mice. (FIG. 2A) Confocal image of pyramidal neurons from the CA1 region of the hippocampus (inset) and 3D visualization of spines from dendritic branch from IMARIS Software were used to quantify spines by class. Scale bar, 10 μm. (FIGS. 2B-2D) Each dot represents the average percent (%) of spines in each class per 10 μm for an individual mouse (n=30; 18 months) that were randomly selected across a range of working memory abilities (40-100% correct transitions, % CT). The average % of spines per class was plotted against the % CT, and index of working memory ability. Analysis of the results revealed a significant positive association between the % long spines and working memory performance (cognitive resilience) (FIG. 2B), which coincided with negative association of % stubby spines with working memory (FIG. 2C). These results support findings in rhesus monkeys, where age-related cognitive decline is associated with loss of long spines [19]. These results also parallel changes in spine classes observed in postmortem brain tissue from humans that exhibit cognitive resilience to AD [20].

FIG. 3 shows that the proportion of DO mice resilient to CFM deficits at 24 months (27%) matches estimates in human cohorts (32% [21]). The histogram shows distribution of aged DO mice (24 months) relative to their recall of contextual fear memory (CFM, mean percent freezing). The plot shows 27% mice with robust recall of CFM (range 62%-100%, light gray) compared to that of young wild-type (WT) mice (62%, dashed line) reported previously [22,23].

FIGS, 4A-4E show DLGAP2 is associated with cognitive function and Alzheimer's disease in diverse human populations. (FIG. 4A) The association between DLGAP2 RNA levels measured in postmortem prefrontal cortex tissue and longitudinal changes in global cognitive performance during the years preceding death are shown. Normalized DLGAP2 expression is presented along the x-axis, and annual change in global cognitive performance is presented along the y-axis. The shaded area around the regression line represents the 95% confidence interval. (FIG. 4B) The data from FIG. 4A is separated into three groups: normal controls (NC), minor cognitive impairment (MCI), and Alzheimer's disease (AD). The regression lines for each population are shown. (FIG. 4C) Differences in DLGAP2 expression across clinical diagnostic groups defined at the final research visit prior to death. Diagnosis is presented along the x-axis, prefrontal cortex expression of DLGAP2 is presented along the y-axis. ** p21 0.01, * p<0.05. (FIG. 4D) Differences in DLGAP2 expression across clinical diagnostic groups defined at the final research visit prior to death. Diagnosis is presented along the x-axis, entorhinal cortex expression of DLGAP2 is presented along the y-axis. ** p<0.01, * p<0.05. (FIG. 4E) A single nucleotide polymorphism (SNP) located at chr8: 303 1316870 (MAF=0.01) was modestly associated with AD within a GWAS of African-American individuals (p=9.2×10⁻⁵). Current Ensembl annotation places this SNP within the first intron of DLGAP2.

FIG. 5 shows a quantile-quantile plot for the association between DNA methylation pattern from the DLGAP2 region and residual cognition. The observed association of each CpG within the DLGAP2 region with residual cognition was plotted as a quantile-quantile plot, along with the confidence intervals (CI) derived from 10,000 simulated association statistics. Observed associations were stronger than simulated test results (Fisher's method; p=0.038), indicating that the DNA methylation pattern from the DLGAP2 region is associated with residual cognition.

### DETAILED DESCRIPTION

Currently, there is a need to understand mechanisms underlying age-related cognitive decline and increased susceptibility to dementias such as AD. While these conditions are highly heritable, identifying precise genetic variants involved in mediating susceptibility remain difficult in human populations, particularly those under-represented in scientific studies. Genetically diverse populations of mice such as the DO represent ideal tools to inform human studies and prioritize hits in biologically relevant genes that may otherwise be ignored as background statistical noise.

Described herein is a large-scale cross sectional evaluation of cognitive performance in a mouse model from 6 to 18 months of age and the surprising identification of a single protein coding gene, disks large-associated protein 2 (DLGAP2), that likely mediates the observed age-related decline. Further, it is demonstrated that DLGAP2 is associated with age-related cognitive decline and AD in diverse human populations. These results highlight the utility of the mouse model to inform studies in human patients and enable the prioritization of variants for further study. These variants likely would have gone unnoticed without supporting evidence provided by a cross-species analysis. This is particularly important when considering human populations that may be under-represented in scientific studies, where the power and sample size may not be sufficient to isolate genome-wide signal over background statistical noise.

In some aspects, the present disclosure provide methods of contacting a neuronal cell neuron) with an agent that increases the expression of DLGAP2 or the activity of DLGAP2 (increases DLGAP2 expression and/or activity), a gene identified herein as differentially expressed in clinically diagnosed groups (normal cognition, mild cognitive impairment, and Alzheimer's disease). Other aspects of the present disclosure provide methods of delivering to a subject having symptoms of age-related cognitive decline, an agent that increases expression of DLGAP2 or the activity of DLGAP2. In some embodiments, the subject has AD.

Contacting a neuronal cell with an agent includes exposing a neuronal cell (e.g., in vivo or in vitro) to an agent (e.g., a therapeutic agent) such that the neuronal cell comes into physical contact with the agent. For example, the step of contacting a neuronal cell with an agent may include delivering the agent to a composition that includes the neuronal cell, and/or delivering the neuronal cell to a composition that includes the agent. A neuronal cell may also be contacted by an agent when the agent is delivered to a subject in which the neuronal cell is present (e.g., brain).

Delivery of an agent to a subject may be by any route known in art. For example, delivery of the agent may be oral, intravenous (e.g., viral vectors, exosomes), intranasal, intramuscular, intrathecal, or subcutaneous. Other delivery routes may be used.

An agent, in some embodiments, is a therapeutic agent and/or a prophylactic agent. An agent may be a biomolecule or a chemical agent. In some embodiments, an agent is a polynucleotide (e.g., double-stranded or single-stranded DNA or RNA, such as a guide RNA (gRNA) (e.g., in combination with Cas9), messenger RNA (mRNA). or an RNA interference (RNAi) molecule, such as antisense RNA, small interfering RNAs (siRNAs), short hairpin RNAs (shRNAs), and/or microRNAs (miRNAs)). In some embodiments, an agent is a polypeptide (e.g., protein and/or peptide). Non-limiting examples of polypeptides include antibodies (e.g., monoclonal antibodies and/or antibody fragments, such as single change variable fragments (scFvs)). An agent, in some embodiments, is a cellular agent, such as a stem cell (e.g., pluripotent stem cell, such as an induced pluripotent stem cell). In some embodiments, an agent is small molecule drug (e.g., chemical compound).

An agent is considered to increase expression of a gene (e.g., DLGAP2)if expression of the gene is increased following exposure of the agent to a neuronal cell comprising the gene. In some embodiments, the change in gene expression is relative to a control, such as gene expression from a neuronal cell not exposed to the agent. In some embodiments, an agent increases expression of a gene by at least 10%, at least 20%, at least 30%, at least 40%, at least 50%, at least 60%, at least 70%, at least 80%, at least 90%, or at least 100% (e.g., by 10%-100%), relative to a control.

Likewise, an agent is considered to increase activity of a product (e.g., DLGAP2 protein) encoded by a gene if activity of the product is increased following exposure of the agent to a neuronal cell comprising the gene encoding the protein. In some embodiments, the change in activity is relative to a control, such as activity in a neuronal cell not exposed to the agent. In some embodiments, an agent increases activity of a product by at least 10%, at least 20%, at least 30%, at least 40%, at least 50%, at least 60%, at least 70%, at least 80%, at least 90%, or at least 100% (e.g., by 10%-100%), relative to a control.

In some embodiments, an agent increases expression of a gene (e.g., DLGAP2) by at least 1.5-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, or at least 20-fold (e.g., 1.5 fold-20-fold).

Methods of assessing whether an agent decreases or increases expression and/or activity of a particular gene and/or protein, such as DLGAP2 are known, any of which may be used to identify an agent that modulates DLGAP2 expression and/or activity (e.g., small molecule inhibitor screening (e.g., Yip K. W., Liu F F. (2011) Small Molecule Screens. In: Schwab M. (eds) Encyclopedia of Cancer. Springer, Berlin, Heidelberg), RNA interference design (e.g., Reynolds, A., Leake, D., Boese, Q. et al. Rational siRNA design for RNA interference. Nat Biotechnol 22, 326-330 (2004)), production of antibodies, e.g., monoclonal antibodies (e.g., VxP Biologics, Patheon, Pacific Immunology, ProMab,, BxCell), etc.).

Neuronal cells (e.g., human neuronal cells or rodent neuronal cells) include neurons, Other brain cell types are encompassed by the present disclosure, including, for example, neuroglia (e.g., oligodendrocytes, microglia, and astrocytes). Examples of neuronal cells include Purkinje cells, granule cells, motor neurons, tripolar neurons, pyramidal cells, chandelier cells, spindle neurons, and stellate cells. In some embodiments, a neuronal cell (neuron) is present in the hippocampus (e.g., hippocampal long spines), cortex, or cerebellum. Neurons of the present disclosure, in some embodiments, are used to test the function of an agent (e.g., in vitro), for example, the extent to which (if any) and agent modifies (e.g., increases) expression of a gene or activity of a product encoded by a gene as provide herein. Thus, in some embodiments, neurons (e.g., in vitro or in an in vivo mouse model) may be modified (e.g., genomically modified) to express or overexpress (e.g., knock in) expression of DLGAP2 (or upstream or downstream genes) as provided herein.

A subject may be a human subject or a rodent (e.g., mouse model). In some embodiments, a subject is a transgenic mouse that expresses or overexpresses (e.g., knock in) DLGAP2 (or upstream or downstream genes). In some embodiments, the subject is a human subject, for example, a subject having (e.g., diagnosed with and/or exhibiting symptoms of) age-related cognitive decline. In some embodiments, the human subject has (e.g., is diagnosed with and/or exhibits symptoms of) Alzheimer's disease.

### Age-Related Cognitive Decline and Alzheimer's Disease

In some aspects, the present disclosure provides a method of delivering to a subject having cognitive decline an agent that modifies the expression of DLGAP2. Age-related cognitive decline is a disorder of the brain. Manifestations of age-related cognitive decline include abnormal structure(s), function(s), or other process(es) in the brain. Age-related cognitive decline refers to a reduced level or loss of cognitive function, including, for example, one or more of the following functions: higher reasoning, memory, concentration, intelligence, and other reductions in mental functions.

The severity of age-related cognitive decline can range from mild cognitive impairment (MCI) to advanced dementia. Alzheimer's disease (AD) is the most common form of dementia, a term that encompasses memory loss and other intellectual abilities serious enough to interfere with the activities of daily life.

In some embodiments, a subject has a mild cognitive impairment. In some embodiments, a subject has MCI. In some embodiments, a subject has dementia. In some embodiments, a subject has AD.

Management of AD includes maintaining quality of life, maximizing function in daily activities, enhancing cognition/mood/behavior, fostering a safe environment, and promoting social engagement. While there is no cure for AD, medications and various management strategies are used to temporarily improve symptoms and to slow the progression of the disease. Medications that may be used are directed to cognitive enhancement (e.g., improving mental function, lowering blood pressure, and balancing mood), and include Donepezil, Galantamine, Memantine, and Rivastigmine. Any of the foregoing medications may be used in combination with agents that increase DLGAP2 expression and/or activity.

Cognitive changes during aging result from changing brain chemistry, for example changes in neurons. Over time, neurons throughout the brain decrease in size and number of synaptic connections. The population of neurons also decreases. The reduction in synaptic density is particularly detrimental to cognitive function. AD, in particular, is characterized by a loss of synapses and neurons in the cerebral cortex and other areas of the brain, as well as the accumulation of extracellular protein-containing deposits (amyloid plaques) and neurofibrillary tangles (tau tangles). Plaques are dense deposits of beta-amyloid peptide and cellular material located outside and around neurons. Tangles comprise aggregates of microtubule-associated tau protein. The tau protein becomes hyperphosphorylated and accumulates within the neurons themselves. The neurons impacted by the plaques and tangles then lose their respective synaptic connections with other neurons, and may die. Thus, in some embodiments, neurons of the cerebral cortex are contacted with an agent that increases DLGAP2 expression and/or activity, for example, in an amount that reduces accumulation of beta-amyloid peptide and/or tau protein.

Symptoms of age-related cognitive decline include decrease in processing speed (e.g., speed at which cognitive activities are performed, speed of motor responses), attention (e.g., ability to concentrate and focus on specific stimuli), memory (e.g., episodic memory, semantic memory), visuospatial constructions, and executive functioning (e.g., the ability to engage in independent, appropriate, purposive, behavior). Symptoms associated with AD, in particular, include behavioral changes (e.g., aggression, agitation, difficulty with self-care, irritability, personality changes, restlessness, lack of restrain, wandering, becoming lost), mood changes (e.g., anger, apathy, general discontent, loneliness, mood swings), psychological changes (e.g., depression, hallucinations, paranoia), as well as several miscellaneous symptoms, including the inability to combine muscle movements, jumbled speech, and loss of appetite. Risk factors for cognitive decline, including AD, may include diabetes, mid-life obesity, mid-life hypertension, hyperlipidemia, smoking status, diet, physical activity, alcohol consumption, cognitive training, social engagement, traumatic brain injury, depression, and lack of sleep.

In some embodiments, a subject of the present disclosure exhibits one or more symptoms and/or risk factors of cognitive decline or AD.

Treatment of age-related cognitive decline includes, in some embodiments, alleviating symptoms of age-related cognitive decline. Alleviation of age-related cognitive decline refers to the process of making the symptoms of cognitive decline less intense and/or more bearable.

In some aspects, neurons of a subject having symptoms of age-related cognitive decline exhibit aberrant expression (e.g., decreased expression) of DLGAP2 compared to a subject not having symptoms of cognitive decline.

In some aspects, neurons of a subject having symptoms of age-related cognitive decline exhibit aberrant activity (e.g., decreased expression) of DLGAP2 compared to a subject not having symptoms of cognitive decline.

### DLGAP2

In some aspects, the present disclosure provides methods of delivering to a neuronal cell (neuron) or to a subject (e.g., having symptoms of cognitive decline and/or having AD) an agent that modifies the expression of a DLGAP2 or the activity of a product encoded by (e.g., DLGAP2 protein) a DLGAP2 differentially expressed by neurons, as provided herein. The disks large-associated protein 2 (DLGAP2)(Gene ID: 9228) gene encodes the DLGAP2 protein. The DLGAP2 protein is a membrane-associated guanylate kinase localized to the postsynaptic density in neuronal cells. The kinase is part of a family of signaling molecules expressed at various submembrane domains and contains the PDZ, SH3 and the guanylate kinase domains. DLGAP2 may play a role in the molecular organization of synapses and in neuronal cell signaling. As described herein, decreases in DLGAP2 are associated with age-related cognitive decline and AD in diverse populations. Thus, low levels of DLGAP2 expression and/or activity may be indicative of age-related cognitive decline.

### Pathway Genes

In some aspects the present disclosure provides methods comprising contacting a neuronal cell with an agent that modifies expression of or modifies activity of a product encoded by a pathway gene upstream from DLGAP2.

In some aspects, the present disclosure provides methods comprising contacting a neuronal cell with an agent that modifies expression of or modifies activity of a product encoded by a pathway gene downstream from DLGAP2.

A pathway gene is an upstream gene or a downstream gene of a biological pathway in which a gene of interest functions. A pathway gene is considered upstream from a gene of interest when the pathway gene has an effect (direct or indirect) on the gene of interest. A pathway gene is considered downstream from a gene of interest when the gene of interest has an effect (direct or indirect) on the pathway gene.

DLGAP2 is part of protein-protein interactions at synapses and is involved in transmission across chemical synapses. Non-limiting examples of genes encoding a protein involved in these interactions include MAGI3, MAGI2, DLGAP1, SHANK1, HOMER3, GRM5, SHANK2, NLGN4X, NLGN4Y, DBNL, SHANK3, NLGN3, GRM5, GRM1, NLGN1, NLGN2, DLG4, GRK5, ADRB1, and NOS1. Thus, in some embodiments, an agent of the present disclosure modifies increases or decreases) expression of or modifies (e.g., increases or decreases) activity of a product encoded by one or more genes selected from MAGI3, MAGI2, DLGAP1, SHANK1, HOMER3, GRM5, SHANK2, NLGN4X, NLGN4Y, DBNL, SHANK3, NLGN3, GRM5, GRM1, NLGN1, NLGN2, DLG4, GRK5, ADRB1, and NOS1.

### Post-Translational Modifications

In some embodiments, an agent used as provided herein affects post-translational modification of DLGAP2 protein. Post-translational modification of proteins refers to the chemical changes proteins may undergo after translation. Such modifications come in a wide variety of types, and are mostly catalyzed by enzymes that recognize specific target sequences in specific proteins, The most common modifications are the specific cleavage of precursor proteins; formation of disulfide bonds; or covalent addition or removal of low-molecular-weight groups, thus leading to modifications such as acetylation, amidation, biotinylation, cysteinylation, deamidation, farnesylation, formylation, geranylgeranylation, glutathionylation, glycation (nonenzymatic conjugation with carbohydrates), glycosylation (enzymatic conjugation with carbohydrates), hydroxylation, methylation, mono-ADP-ribosylation, myristoylation, oxidation, palmitoylation, phosphorylation, poly(ADP-ribosyl)ation, stearoylation, or sulfation.

In some embodiments, an agent may affect methylation of a DLGAP2 protein, for example, by directly methylating the protein or causing another agent (e.g., enzyme) to methylate a DLGAP2 protein.

### EXAMPLES

The present disclosure is further illustrated by the following Examples. These Examples are provided to aid in the understanding of the disclosure, and should not be construed as a limitation thereof.

### Example 1. Cross-Species Analyses Identify DLGAP2 as a Mediator of Age-Related Cognitive Decline and Alzheimer's Disease

### Dlgap2 Mediates Cognitive Longevity in Diversity Outbred Mice

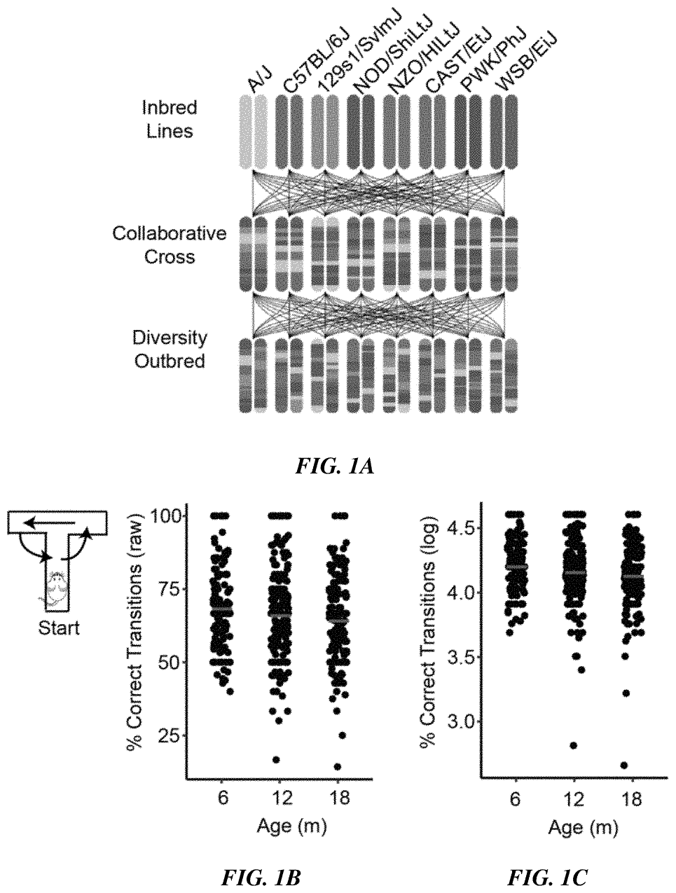

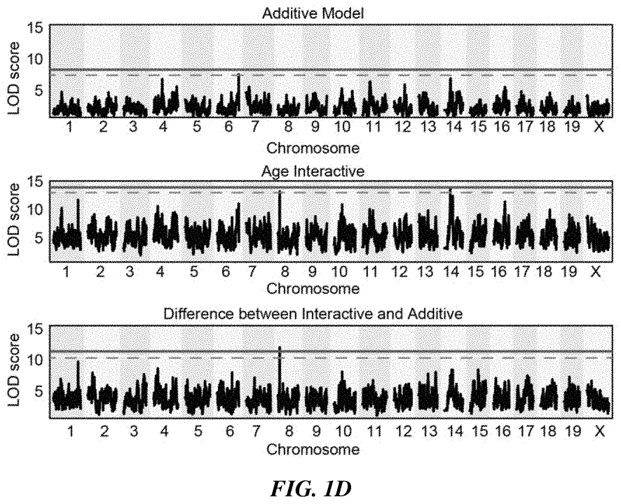

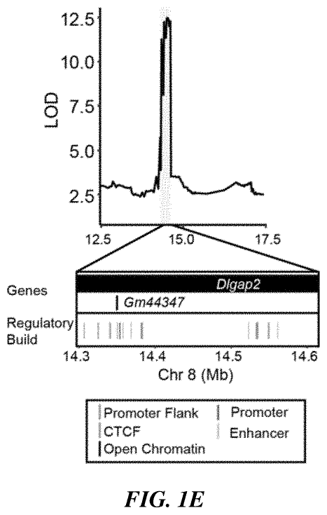

The genetic diversity represented in the Diversity Outbred (DO) mouse population (FIG. 1A) was used to identify precise genes involved in mediating cognitive function in aging. Working memory was evaluated on the T-maze [11] at either 6, 12, or 18 months in 487 DO mice (6m=66F/67M, 12m=102F/96M, 18m=76F/80M. All groups performed above chance (50%, FIG. 1B), and the data was log transformed for subsequent genetic mapping (FIG. 1C). A slight but significant effect of age was observed [F(2, 284)=3.2, p=0.04] across DO mice. Genetic mapping in r/qtl2 identified a quantitative trait locus (QTL) on chromosome 8 (FIG. 1D) that significantly interacted with age to mediate working memory performance across the lifespan (LOD=12.5, 1.5 LOD interval=14.3-14.6 Mb). A single protein-coding gene, Dlgap2, was found to be located within the QTL interval, along with a number of regulatory elements (FIG. 1E). Given the complicated nature of assigning causality to regulatory elements, and the established role of Dlgap2 as a critical component of the postsynaptic density [12], Dlgap2 was the focus as the top positional candidate.

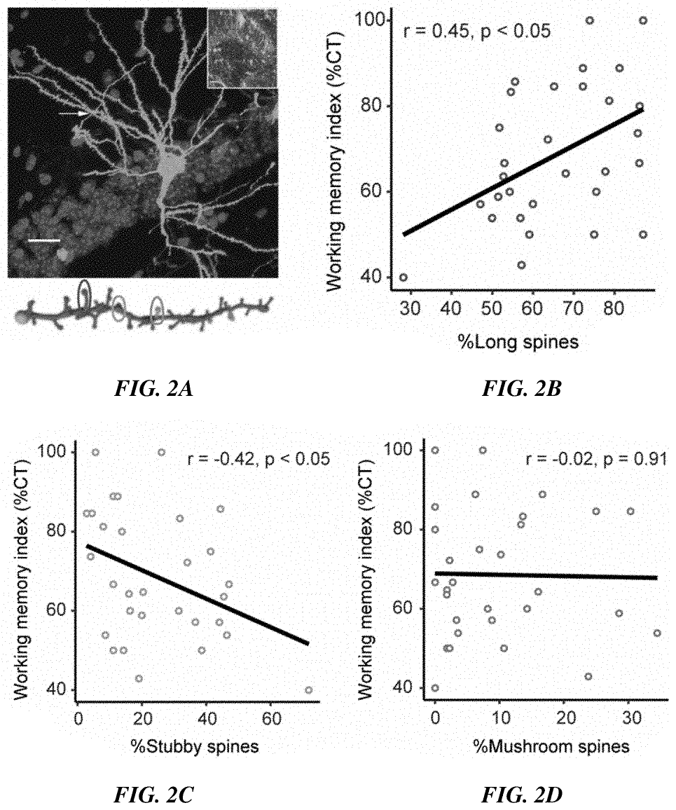

As shown in FIGS. 2A-2D increased density of hippocampal long spines positively correlate with cognitive resilience in aging DO mice. Confocal image of pyramidal neurons from the CA1 region of the hippocampus (inset) and 3D visualization of spines from dendritic branch from IMARIS Software were used to quantify spines by class (FIG. 2A). Analysis of the results revealed a significant positive association between the % long spines and working memory performance (cognitive resilience) (FIG. 2B), which coincided with negative association of % stubby spines with working memory (FIG. 2C). These results support findings in rhesus monkeys, where age-related cognitive decline is associated with loss of long spines [19]. These results also parallel changes in spine classes observed in postmortem brain tissue from humans that exhibit cognitive resilience to AD [20].

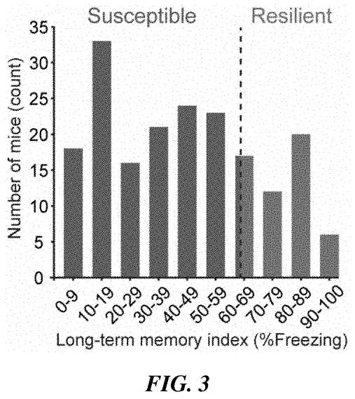

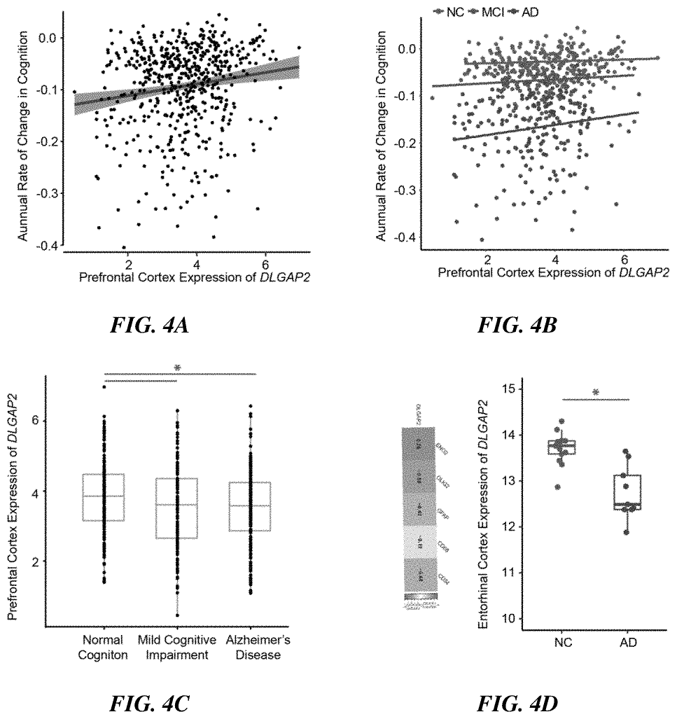

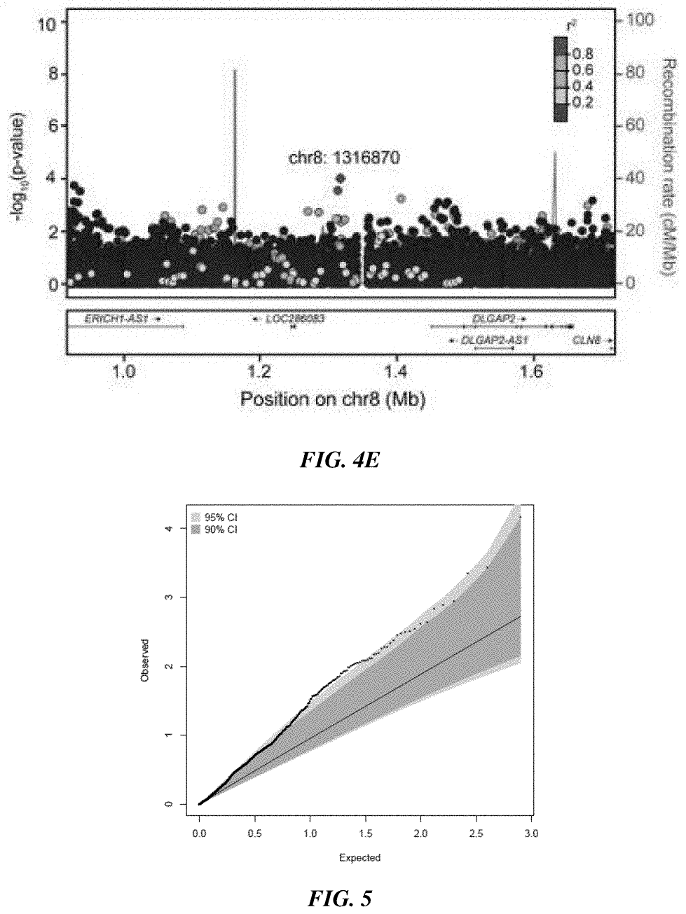

Further, the proportion of DO mice resilient to CFM deficits at 24 months (27%) S matches estimates in human cohorts (32% [21]) (FIG. 3). The histogram in FIG. 3 shows distribution of aged DO mice (24 months) relative to their recall of contextual fear memory (CFM, mean percent freezing). The plot shows 27% mice with robust recall of CFM (range 62%-100%, light gray) compared to that of young wild-type (WT) mice (62%, dashed line) reported previously [22,23].

### DLGAP2 is Associated with Exacerbated Cognitive Decline and AD in Humans

The possible outcome that DO mice are a translationally relevant resource and DLGAP2 is associated with cognitive decline in human populations was tested, The DLGAP2 genotype, and its effect on longitudinal decline were first evaluated on a modified mini mental state exam across elderly women enrolled in the Women's Health Initiative Memory Study. A modest association was observed (p<0.05, data not shown). Next, the functional association of DLGAP2 expression levels on cognition was evaluated, leveraging mRNA data measured in the prefrontal cortex (PFC) as part of the Religious Orders Study/Memory and Aging Project (ROS/MAP) was used. Across the ROS/MAP cohort, higher levels of DLGAP2 expression in the PFC was associated with increased cognitive decline (β=0.002; FIG. 4A). Notably, this association was strongest when considering those individuals with clinically diagnosed AD (FIG. 4B). In addition, DLGAP2 expression was significantly lower in the PFC of participants diagnosed with both mild cognitive impairment (MCI) and clinical AD relative to those with normal cognition (NC) (F(2,528)=4.4, p=0.01; FIG. 4C). As DLGAP2 is a component of synapses [12] and highly correlated with expression of the neuronal marker ENO2 (FIG. 4D, left), it is possible this down-regulation of DLGAP2 is due to neurodegeneration that occurs in MCI and AD. However, when considering only neuronal expression data [13] to control for number of neurons evaluated, a significant down-regulation of DLGAP2 in AD remained (FIG. 4D, right), suggesting reduced DLGAP2 occurs independent of frank neurodegeneration.

### DLGAP2 is Differentially Expressed in Brains of Those with Cognitive Impairment

While not associated with neurodegeneration, we next evaluated whether DLGAP2 was associated with other neuropathological hallmarks of Alzheimer's disease. Lower levels of DLGAP2 were associated with greater β-amyloid load (β=−0.13, p=0.002) and more neurofibrillary tangles (β=−0.11, p=0.02), both measured with IHC. No associations were observed with non-Alzheimer neuropathologies (data not shown).

### Genetic Variants in the DLGAP2 Region are Associated with Alzheimer's Dementia

Given the association between DLGAP2 expression and cognitive decline, we next sought to evaluate whether genetic variants in DLGAP2 were associated with risk for clinically diagnosed Alzheimer's dementia. We evaluated SNPs within the DLGAP2 region (±50 Kb) within published and pending GWAS studies. Among individuals with European ancestry [13], one locus just downstream of DLGAP2 was associated with Alzheimer's dementia (top SNP rs2957061, p=3.6×10⁻⁵. Among African American individuals, a locus within DLGAP2 was associated with Alzheimer's dementia (top SNP chr8:1316870, MAF=0.01, p=9.2×10⁻⁵. Additionally, there is evidence that variants in the DLGAP2 region causally influence DLGAP2 expression, as rs111865014 has been identified as a cis-eQTL in hippocampal tissue from 171 over 1000 non-diseased individuals as part of the Genotype-Tissue Expression project (GTEx, p=9.7×10⁻⁷). Together, these results suggest DLGAP2, both at the variant and expression level, is causally involved in disease pathogenesis.

### DLPFC Methylation of DLGAP2 is Associated with Residual Cognitive Performance

A previous GWAS [14] reported that rs34130287C, a SNP within the first intron of DLGAP2, was suggestively associated with worse residual cognition (p=4.0×10⁻⁶), a trait that quantified the gap between cognitive performance after regressing out the effect of neuropathology. DLGAP2 was not pursued as a potential candidate because NCBI and Ensembl annotations, at the time of prior report, did not include rs34130287C within DLGAP2. However, current annotations place this SNP within DLGAP2. Using the same dataset and methods as initially reported [14], we observed a significant relationship between the overall methylation pattern of the DLGAP2 region in DLPFC and residual cognition (p:=0.038; FIG. 5). As methylation at the Dlgap2 locus has been shown to influence Dlgap2 expression in mouse [15], we hypothesize this effect is mediated by alterations in DLGAP2 expression in the DLPFC.

### Utility of Diversity Outbred Mice for Cross-Species Analyses

With the increasing accessibility of genomic technologies, the number of genome-wide association studies (GWAS) exploring the genetic mechanisms underlying complex traits has drastically increased. However, all populations have not benefitted from these scientific advances equally. A number of factors contribute to this under-representation, including logistical (lack of access to medical centers), systemic (bias toward utilizing data from existing cohorts), and cultural decisions to avoid participation. While initiatives are underway across the globe to include more diverse populations in genomic studies, it will take years or even decades before equal representation is achieved GWAS. As a result, population-specific genetic mechanisms underlying diseases, and treatments that may prevent or cure them, remain undiscovered, largely due to a lack of statistical power. To better inform population-specific analyses, mouse studies offer a powerful way to prioritize candidates. In particular, the DO population provides an advantage over previous genetically diverse resources, including a higher degree of genetic diversity, smaller haplotype blocks leading to more precise genomic mapping, and therefore fewer putative candidates to test for translational relevance [5]. Combined with the reproducible recombinant inbred strains from the related CC panel [15], candidate genes nominated by studies in the DO have the potential to greatly contribute to understanding of genetic mechanisms underlying complex traits in both mouse and humans.

### DLGAP2 as a Mediator of Cognitive Decline

DLGAP2, also known as SAPAP2 or GKAP2, is one of the main components of postsynaptic density scaffolding proteins and plays a critical role in synaptic function [16]. Mutant mice that lack Dlgap2 show impaired initial reversal learning, reduced spine density in the frontal cortex, and deficits in synaptic communication [16]. Together, these results provide a mechanistic explanation by which cognitive decline may be exacerbated in aged DO mice as well as humans with MCI and AD with reduced DLGAP2. Dendritic spines are critically involved in neuronal function, as changes in spine type, size, and morphology allow dynamic control of receptor density, electrical resistance, and local transcription and translation at the synapse [17]. Maintenance of spines has been observed to associate with cognitive resilience to AD pathology [18], and a robust loss of synapses has been observed in AD, both after post-mortem evaluation and through in vivo PET imaging studies [17]. However, mechanisms underlying this loss of synapses are still poorly understood. As demonstrated herein, DLGAP2 is a likely driver of cognitive decline and later transition to dementia, potentially mediated by a loss of synapses. One possible outcome is that genetic variants in DLGAP2 increase susceptibility to spine loss and cognitive decline.

### Materials and Methods

### Genetic Mapping

Genetic mapping was conducted according to established procedures (refs). R/qtl2 was used to perform single quantitative trait loci (QTL) scans with sex and age as covariates. To identify QTL that interact with age, age was included as an interactive covariate. Permutation tests were used to evaluate significance. Genes in the 1.5 LOD confidence interval were identified using the biomaRt package.

### Participants

To validate the translation relevance of candidate genes associated with memory performance in DO mice, data was obtained from a number of well-defined cohorts of cognitive aging and AD. Two cohort studies of cognitive aging, The Religious Orders Study (ROS) and The Rush Memory and Aging Project (MAP), both enrolled participants free of dementia who agreed to annual clinical evaluations and brain donation at death. Informed written consent was obtained from all participants, and all research adhered to individual Institutional Review Board (IRB)-approved protocols.

### Quantification of Cognitive Function

In ROS/MAP participants, cognitive function was quantified into a single composite measure generated by averaging the z-scores of 17 cognitive tests that spanned 5 domains of cognitive function (episodic, semantic, and working memory, perceptual orientation, and perceptual speed) (Wilson R S, et al. Neurology 2015; 85(11):984-991).

### Measures of Gene Expression in Human Brain Tissue

In ROS/MAP participants, RNA expression levels were extracted from frozen, manually dissected dorsolateral prefrontal cortex (PFC) tissue (Lim A S, et al. PLoS genetics 10: e1004792). As previously described (ref), isolation of RNA was performed using the RNeasy lipid tissue kit (Qiagen, Valencia., Calif.) and it was reverse transcribed using the Ilumina® Total Prep™ RNA Amplification Kit from Ambion (Illumina, San Diego, Calif.). Processing of the expression signals was performed using the BeadStudio software suite (Illumina, San Diego, Calif.). Standard normalization and quality control methods were then employed, as previously described (Lim A S, el al. PLoS genetics 10: e1004792).

### Statistical Analyses

Statistical analyses were completed using RStudio (version 1.1.453). Longitudinal associations between DLGAP2 expression and global cognition in ROS/MAP were tested using mixed-effects regression. Age at death, sex, gene expression level, and an interval term (ie, time in years prior to death) were considered fixed effects, and the intercept and a gene expression x interval interaction term were considered random effects. Differences in gene expression among clinical diagnostic categories in ROS/MAP was performed. Single-SNP associations in African Americans were performed in PLINK (version 1.9) using linear regression, an additive genetic model, and covarying.

### REFERENCES

1. Reynolds, C. A. and D. Finkel, A meta-analysis of heritability of cognitive aging: minding the “missing heritability” gap.Neuropsychol. Rev, 2015. 25(1): p. 97-112.
2. Deary, I. J., et al., Genetic contributions to stability and change in intelligence from childhood to old age. Nature. 2012. 482(7384): p. 212-5.
3. Harris, S. E. and I. J. Deary, The genetics of cognitive ability and cognitive ageing in healthy older people. Trends Cogn Sci, 2011. 15(9): p. 388-94.
4.Ridge, P. G., et al., Alzheimer's disease: analyzing the missing heritability. PLoS One, 2013. 8(11): p. e79771.
5. Churchill, G. A., et al., The Diversity Outbred mouse population. Mamm Genome, 2012. 23(9-10): p. 713-8.
6. Logan, R. W., et al., High-precision genetic mapping of behavioral traits in the diversity outbred mouse population. Genes Brain Behav, 2013. 12(4): p. 424-37.
7. Smallwood, T. L., et al., High-resolution genetic mapping in the diversity outbred mouse population identifies Apobec1 as a candidate gene for atherosclerosis. G3 (Bethesda), 2014. 4(12): p. 2353-63.
8. Chester, E. J., Out of the bottleneck: the Diversity Outcross and Collaborative Cross mouse populations in behavioral genetics research. Mamm Genome, 2014, 25(1-2): p. 3-11.
9. Philip, V. M., et al., Genetic analysis in the Collaborative Cross breeding population. Genome Res, 2011. 21(8): p, 1223-38.
10. Srivastava, A., et al, Genomes of the Mouse Collaborative Cross. Genetics, 2017, 206(2): p. 537-556.
11.Wenk, G. L., Assessment of spatial memory using the T maze. Curr Protoc Neurosci, 2001. Chapter 8: p. Unit 8 5B.
12. Li, J., et al., Spatiotemporal profile of postsynaptic interactomes integrates components of complex brain disorders. Nat Neurosci, 2017. 20(8): p. 1150-1161.
13. Liang, W. S., et al., Alzheimer's disease is associated with reduced expression of energy metabolism genes in posterior cingulate neurons. Proc Natl Acad Sci USA, 2008. 105(11): p. 4441.-6.
14. Popejoy, A. B. and S. M. Fullerton, Genomics is failing on diversity. Nature, 2016. 538(7624): p. 161-164.
15. Churchill, G .A., e al., The Collaborative Cross, a community resource for the genetic analysis of complex traits. Nat Genet, 2004. 36(11): p. 1133-7.
16. Jiang-Xie, L. F., et al., Autism-associated gene Dlgap2 mutant mice demonstrate exacerbated aggressive behaviors and orbitofrontal cortex deficits. Mol Autism, 2014. 5: p. 32.
17. Dorostkar, M. M., et al., Analyzing dendritic spine pathology in Alzheimer's disease: problems and opportunities. Acta Neuropathol, 2015. 130(1): p. 1-19.
18. Boras, B. D., et al., Dendritic spines provide cognitive resilience against Alzheimer's disease. Ann Neurol, 2017. 82(4): p. 602-614.
19. Dumitriu, D. et al. Selective changes in thin spine density and morphology in monkey prefrontal cortex correlate with aging-related cognitive impairment. J Neurosci 30, 7507-7515, doi:10.1523/JNEUROSCI.6410-09.2010 (2010).
20. Bows, B. D. et al. Dendritic spines provide cognitive resilience against Alzheimer's disease. Ann Neural 82, 602-614, doi:10.1002/ana.25049 (2017).
21. Negash, S. et al. Resilient brain aging: characterization of discordance between Alzheimer's disease pathology and cognition. Curr Alzheimer Res 10, 844-851 (2013).
22. Kaczorowski, C. C. & Disterhoft, J. F. Memory deficits are associated with impaired ability to modulate neuronal excitability in middle-aged mice. Learn Mem 16, 362-366, doi:10.1101/1m.1365609 (2009).
23. Gaiteri, C. et al. Identifying robust communities and multi-community nodes by combining top-down and bottom-up approaches to clustering. Sci Rep 5, 16361, doi:10.1038/srep16361 (2015).

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between the upper and lower ends of the range are specifically contemplated and described herein.

1. A method comprising delivering to a subject an agent that modulates disks large-associated protein 2 (DLGAP2) expression and/or activity, wherein the subject has symptoms of age-related cognitive decline.

2. The method of claim 1, wherein the agent increases DLGAP2 expression and/or activity.

3. The method of claim 1, wherein the agent affects post-translational modification of DLGAP2 protein.

4. The method of claim 3, wherein the post-translational modification is selected from methylation, phosphorylation, glycosylation, acetylation, and amidation.

5. The method of claim 4, wherein post-translational modification is methylation.

6. The method of claim 1, wherein the subject has a modification in a DLGAP2 gene.

7. The method claim 1, wherein the subject is a human subject.

8. The method of claim 7, wherein the human subject has Alzheimer's disease.

9. The method of claim 1, wherein the agent is delivered in an amount effective to alleviate the symptoms of the age-related cognitive decline.

10. The method of claim 1, wherein the agent is delivered in an amount effective to slow or stop progression of the age-related cognitive decline.

11. The method of claim 1, wherein the agent is selected from polypeptides, polynucleotides, small molecule drugs.

12. A method comprising delivering to a subject an agent that modulates expression of, or modulates activity of, a product encoded by a pathway gene upstream from or downstream from DLGAP2, wherein the subject has symptoms of age-related cognitive decline.

13. The method of claim 12, wherein the agent increases expression of, or increases activity of, a product encoded by a pathway gene upstream from or downstream from DLGAP2

14. The method of claim 12, wherein the subject is a human subject.

15. The method of claim 14, wherein the subject has Alzheimer's disease.

16. The method of claim 1, wherein the agent is delivered in an amount effective to alleviate the symptoms of the age-related cognitive decline.

17. The method of claim 1, wherein the agent is delivered in an amount effective to slow or stop progression of the age-related cognitive decline.

18. The method of claim 1, wherein the agent is selected from polypeptides, polynucleotides, small molecule drugs.

19.-21. (canceled)

22. A method comprising administering to a Dlgap2 mutant mouse a candidate agent that modulates DLGAP2 expression and/or activity, and optionally assaying the mouse for an improvement in a symptom of age-related cognitive decline and/or assaying the mouse for an adverse effect.

23.-25. (canceled)
